(19) United States
(12) Patent Application Publication
(10) Pub. No.: US 2019/0252080 A1
(43) Pub. Date: Aug. 15, 2019

(54) VIRTUAL REALITY AND AUGMENTED REALITY GROUP THERAPY FOR TREATING MENTAL HEALTH, SUBSTANCE ABUSE, AND DEVELOPMENTAL DISORDERS

(71) Applicant: IFGCure Holdings, LLC, Los Angeles, CA (US)

(72) Inventor: Stephen H. LIU, Los Angeles, CA (US)

(21) Appl. No.: 16/236,850

(22) Filed: Dec. 31, 2018

Related U.S. Application Data
(60) Provisional application No. 62/612,393, filed on Dec. 30, 2017.

(51) Int. Cl. G16H 80/00; G09B 9/00; A61B 5/16; G06F 3/14

(52) U.S. Cl. CPC G16H 80/00; G06F 3/1454; A61B 5/167; G09B 9/00

(57) ABSTRACT

Processes, systems, methods, and software that use virtual reality in the mental health, substance abuse, and/or developmental disorder field specifically for the purpose of group therapy. More particularly, the processes systems, methods, and software use augmented and virtual reality (AR/VR) to allow licensed healthcare professionals to conduct group therapy sessions for mental health, substance abuse, and/or developmental disorder patients.

### CROSS REFERENCE TO RELATED APPLICATIONS

This application claims the benefit of U.S. Provisional Application No. 62/612,393 entitled MENTAL HEALTH, SUBSTANCE ABUSE, AND DEVELOPMENTAL DISORDER TREATMENT USING VIRTUAL REALITY APPLICATIONS AND CONTENT FOR GROUP THERAPY, UTILIZING A THERAPIST OR LICENSED HEALTHCARE PROFESSIONAL PRESENT WITHIN THE VIRTUAL ENVIRONMENT FOR TREATMENT, filed Dec. 30, 2017, which is incorporated herein by reference in its entirety.

### FIELD OF THE INVENTION

The present invention is directed to methods and systems for using augmented and/or virtual reality (AR/VR) technology in the mental health, substance abuse, and/or developmental disorder field. More particularly, the inventive method and system provides a mental health, substance abuse, and/or developmental disorder group therapy treatment that introduces the input of a licensed healthcare professional (LHP), such as a therapist, into the field of view of at least two patients while they are utilizing a wearable medical device or wearable device, while providing a VR environment or AR setting for group therapy.

### BACKGROUND OF THE INVENTION

Mental health, substance abuse, and/or developmental disorder problems are a serious worldwide issue, particularly in the United States. There is a lack of easy access for treatment for veterans, patients with mental disorders, and patients with comorbid substance abuse and mental health disorders. Sadly, there remains a strong social stigma associated with mental health that dissuades individuals from seeking help in a public manner or visiting a therapist office. Often patients are reluctant to visit a therapist or healthcare professional in order to receive treatment because such patients do not want to be seen by others seeking treatment or be perceived by the general public and friends as a ‘mental case’ of someone who needs help.

Furthermore, patients suffering from mental health disorders often have difficulty adhering to a schedule and have difficulty remembering or attending therapy sessions.

Moreover, patients suffering from mental health disorders require treatment when they need it most, i.e., when they are in public high pressure environments, and often it is not possible to arrive at a therapist or healthcare professional's office to receive a treatment.

Alternatively, and recently, telehealth options have become available for patients. Telehealth involves the distribution of health-related services and information via electronic device and telecommunication technologies. Patients use electronic devices to communicate with licensed healthcare professionals (LHPs), such as therapists, rather than going into an office. Telehealth options can reduce costs and counteract the social stigma of seeking professional treatment for their mental health issues.

With rising public and private interest in mental health and overall wellbeing, more patients are seeking treatment—leading to a lack of LHP and/or therapist supply along with an ever-rising demand for therapists. Patients are struggling to gain personal attention from their therapists and it is difficult for patients to access therapists on demand and when needed.

One solution to this problem is to place patients in group therapy or a group therapy environment if indicated for a patient. Group therapy provides similar benefits as one-on-one therapy and has emerged as a popular option as one or more therapists are able treat multiple patients in a group setting, thus providing more efficient and timely care to patients.

In addition, group therapy has benefits on top of individual therapy sessions such as cheaper cost, sharing of their struggles with similar individuals; ability to identify with others; receiving and giving support to others; getting a variety of viewpoints; and seeing successful models of individuals who have gone through similar experiences

Despite the benefits of group therapy, however, there is still a shortage of therapists to provide group therapy sessions. It can also be difficult to corral a group of individuals to all meet at a certain time and place on a standardized schedule, further negatively impacting patient compliance.

Given the issues listed above, there exists a need to both encourage patients to attend group therapy sessions and for the therapy sessions themselves to improve.

Known systems and methods that use telehealth include US20040197750; US20050216243; US20070027406; US20070123757; US20070166690; US20080096665; US20080199841; US20080319252; US20100121156; US20110118555; US20110213197; US20110283203; US20120330869; US20130246084; US20140058755; US20140222462; US20140243608; US20140330576; US20150133812; US20150174362; US20160134840; US20160155352; US20160246936; US20160317781; US20170154159; US20170169177; US20170213006; US20170286617; US20170323485; US20180039752; US20180120947; US20180226158; US20180268110; U.S. Pat. Nos. 5,722,418; 5,736,986; 5,807,114; 6,012,926; 6,057,846; 6,205,716; 7,024,398; 9,576,106; 9,585,616; 9,589,475; 9,649,469; 9,694,155; 9,907,730; 9,978,288; 10,013,531; WO0062850A1.

Despite the proliferation of telehealth, individuals may lack a certain connection or sensory rapport when speaking to their LHP and/or therapist through an electronic device. Retention rates are also of issue with patients often dropping out before completion of treatment using standard telehealth options.

These problems are exacerbated when dealing with patients having mental health, behavioral, or developmental disorder issues. These patients are extremely susceptible, have various issues, and often turn to drugs and other substances in order to solve their problems. Existing telehealth options are not geared towards mental health patients and there is no existing method or system that provides for an adequate solution to treat mental health disorders via telehealth.

Thus, it is an object of the present invention to improve upon present standard telehealth options and provide methods and systems that solve the problems set forth above, specifically for patients having mental health, behavioral and developmental disorders.

What is desired is to improve upon and further exploit present standard telehealth options and provide methods and systems that solve the problems set forth above.

### SUMMARY OF THE INVENTION

It is an object of the present invention to improve upon present standard telehealth options, specifically for patients having mental health, behavioral and developmental disorders.

It is an object of the present invention to provide an augmented and/or virtual reality (AR/VR) technology solution to overcome existing deficiencies in telehealth, specifically using group therapy.

It is an object of the present invention to provide AR or VR in the mental health, substance abuse and/or developmental disorder treatment field specifically for the purpose and facilitation of group therapy.

It is an object of the present invention to provide methods, systems and processes that use a wireless wearable device to deliver to patients an AR/VR environment in a group setting.

It is an object to provide an LHP or therapist inside an AR/VR environment. Inside this environment, the therapist can interact with patients in a way that helps treat their specific mental health, substance abuse and/or developmental disorder problems.

It is an object to provide an LHP or therapist in an AR/VR environment as a character asset that the real-life therapist may speak through. This asset may range from a stock asset character to an identical and realistic figure of the therapist.

It is an object of the present invention to eliminate the need for a patient to travel to a hospital or medical center for mental health, substance abuse, and/or developmental disorder treatment in a group setting, and to protect the confidentiality of the patient so that he or she will not be stigmatized. Also, by being more salient, stimulating, and non-threatening as well as providing comfort and ease of accessibility in most locations, patients are more likely to stick with the entire course of treatment. This can lead to increased engagement and lower no-show rates.

It is an object of the present invention to provide patient ease of use by removing the need to travel outside the home and by substituting handheld technology for wearable technology and may be used in a non-stigmatized fashion from the wearer's home or similar private locations.

It is an object of the present invention to provide a system and method of and for telehealth services whereby patients can interact with their therapists and with other patients in a non-office group setting.

Patients would put on either a wearable device (such as a VR-enabled head mounted display) or wearable medical device in order to receive treatment. For VR group therapy, the therapist and patients would all be inside of a virtual environment and able to interact with each other. For AR group therapy, the therapist and patients would be a projection onto the real world with the ability to interact with each other. These therapists or LHPs would help treat mental health, substance abuse, and/or developmental disorder problems.

These and other objects of the instant invention are achieved by providing a method for treating at least two patients using AR/VR, the method comprising: administering a version of an LHP or therapist and at least two other patients within a field of view or virtual reality environment of both at least two patients and the LHP or therapist.

In certain embodiments, the AR/VR version of an LHP or therapist provides a therapy treatment to the at least two patients.

In certain embodiments, the AR/VR version of an LHP or therapist includes, but is not limited to computer generated inputs, or sensory input such as sound, video, graphics, haptics or GPS data.

In certain embodiments, the AR/VR version of a patient includes, but is not limited to computer generated inputs, or sensory input such as sound, video, graphics, haptics or GPS data.

In certain embodiments, the AR/VR version of an LHP or therapist decreases the likelihood that a patient will dropout and cease therapy.

In certain embodiments, the LHP or therapist is actively treating the at least two patients while the patients are receiving the therapy treatment.

In certain embodiments, the at least two patients receive the therapy treatment outside of an office of the LHP or therapist.

In certain embodiments, a patient receives the therapy treatment treated at a home of the patient.

In certain embodiments, the patient is suffering from a mental health, substance abuse and/or developmental disorder.

Other objects of the invention are achieved by providing a process for mental health, substance abuse and/or developmental disorder patients to seek group therapy or treatment outside of the office, thus encouraging higher rates of therapy usage as well as higher success rates of treatment completion.

Other objects of the invention are achieved by providing a system for treating a patient in group therapy using AR/VR, the system comprising: a processor; software executing on the processor that provides an AR/VR version of a LHP or therapist and at least two patients within a field of view of the patients.

Other objects of the invention are achieved by providing a computer-implemented method for treating patients via group therapy, the method comprising: providing at least three augmented reality (AR) or virtual reality (VR) devices, the devices each having at least one processor; creating, via software executing on a processor, an field of view; overlaying, via software executing on a processor, a therapist within the field of view to create a modified field of view; and displaying the modified field of view on the at least three devices, wherein the therapist within the modified field of view provides a therapy to at least two patients suffering from a mental health, behavioral health, or developmental disorder.

Other objects of the invention are achieved by providing an augmented reality (AR) or virtual reality (VR) system for treating at least two patient suffering from a mental health, behavioral health, or developmental disorder via group therapy, the system comprising: at least three augmented reality (AR) or virtual reality (VR) devices, the devices each having at least one processor; at least one computer-readable storage media storing instructions which, when executed by the at least one processor of each device: creates an field of view, overlays an therapist within the field of view to create a modified field of view, and displays modified field of view on the device, wherein the therapist within the modified field of view provides a therapy to at least two patients suffering from a mental health, behavioral health, or developmental disorder.

Other objects of the invention and its particular features and advantages will become more apparent from consideration of the following drawings and accompanying detailed description. It should be understood that the detailed description and specific examples, while indicating the preferred embodiment of the invention, are intended for purposes of illustration only and are not intended to limit the scope of the invention.

### BRIEF DESCRIPTION OF THE DRAWINGS

FIG. 1 depicts a flowchart detailing an embodiment of the invention detailing a method of VR/AR group therapy, where patients being treated for group therapy targeting mental health, substance abuse, and/or developmental disorders have an AR/VR environment with a therapist inside the environment; and details the same therapist conducting typical group therapy.

FIG. 2 depicts a flowchart of a group therapy session of an embodiment of the invention.

FIG. 3 depicts a flowchart of AR/VR Avatars.

FIG. 4 is a schematic diagram of one embodiment of a system that can be used to practice aspects of the present invention.

FIG. 5 is a flowchart of a therapist with VR patients such that the therapist and VR patients are in a group therapy environment.

### DETAILED DESCRIPTION OF THE INVENTION

This application incorporates by reference U.S. Ser. No. 16/224,778 entitled AUGMENTED REALITY THERAPY FOR TREATING MENTAL HEALTH AND DEVELOPMENTAL DISORDERS filed on Dec. 18, 2018 in its entirety.

In the following description, numerous details are set forth for the purpose of explanation. However, one of ordinary skill in the art will realize that the invention may be practiced without the use of these specific details.

The invention is now described with reference to the drawings, wherein like reference numerals are used to refer to like elements throughout. In the following description, for purposes of explanation, numerous specific details are set forth in order to provide a thorough understanding of the subject invention. It may be evident, however, that the invention can be practiced without these specific details. In other instances, well-known structures and devices are shown in block diagram form in order to facilitate describing the innovation.

As used in this application, the terms “component” and “system” are intended to refer to a computer-related entity, either hardware, a combination of hardware and software, software, or software in execution. For example, a component can be, but is not limited to being, a process running on a processor, a processor, an object, an executable, a thread of execution, a program, and/or a computer. By way of illustration, both an application running on a server and the server can be a component. One or more components can reside within a process and/or thread of execution, and a component can be localized on one computer and/or distributed between two or more computers.

### Augmented Reality

Augmented reality (AR) is an interactive experience of a real-world environment where the objects that reside in the real-world are “augmented” by computer-generated perceptual information, sometimes across multiple sensory modalities, including visual, auditory, haptic, somatosensory, and olfactory.

AR is something different from merely a multimedia system or an interactive computer graphics display. AR involves augmenting a user's vision to introduce or overlay an element. The overlaid sensory information can be constructive (i.e. additive to the natural environment) or destructive (i.e. masking of the natural environment) and is seamlessly interwoven with the physical world such that it is perceived as an immersive aspect of the real environment. In this way, augmented reality alters one's ongoing perception of a real-world environment. Augmented reality is related to two largely synonymous terms: mixed reality and computer-mediated reality. The primary value of augmented reality is that it brings components of the digital world into a person's perception of the real world, and does so not as a simple display of data, but through the integration of immersive sensations that are perceived as natural parts of an environment.

### Virtual Reality

VR is a technological interface that allows users to experience computer-generated environments within a controlled setting; and has been increasingly used in the context of mental health treatment and within clinical research. VR is well suited for use in exposure-based treatment for anxiety disorders as it provides the opportunity for a sense of presence and immersion in a feared environment. VR aims to parallel reality and create a world that is both immersive and interactive. Users fully experience VR when they believe that the paradigm accurately simulates the real-world experience that it attempts to recreate. The sense of presence, or “being there,” in VR is facilitated using technology such as head-mounted displays, gesture-sensing gloves, synthesized sounds, and vibrotactile platforms—which allow for the stimulation of multiple senses and active exploration of the virtual environment. Some VR paradigms are programmed to dynamically react to the actions of the user enabling them to interact and to engage with the VR environment in a more naturalistic and intuitive way. VR is potentially a powerful tool for the psychiatric community because the user experience can be consistently replicated, tested, and modified within a safe environment. VR's precise control of sensory cues, particularly for auditory, tactile, and olfactory systems, increases the sense of realism and memory of the virtual environment.

A “sense of presence” that one experiences in a virtual environment is perhaps the best-known attribute of virtual reality. This appeal to this sense of presence is used to distinguish virtual reality and as something different from merely a multimedia system or an interactive computer graphics display.

### Embodiments of the Invention

In an embodiment of the instant invention, an AR/VR group therapy process method may unfold as follows:

A patient utilizes an AR/VR capable device that may either be a wearable device or a wearable medical device. The AR/VR-capable device may be wirelessly connected to a computer, mobile phone, or similar functioning smart-device apparatus that gives the AR/VR-capable device the capability to project augmented/mixed reality images, namely a therapist or licensed healthcare professional with the capability to interact and speak either through or as the projected image. It is contemplated that the AR/VR-capable device has this same projection capability already built in. A patient may as well wear an auditory system that may or may not be built into the AR/VR-capable device.

The AR/VR-capable device may project a version of a licensed healthcare professional/therapist that is in the patient's field of vision. As may be appreciated, the patient may be able to see the same space before and after using the AR/VR device, while the input of the therapist is anchored to the real world through a projected image. This projection can be used in private places, such as at home or at a friend's house. The licensed healthcare professional/therapist may interact, via the projection, with the patient in order to alleviate mental health, substance abuse, and/or developmental disorders. This interaction and experience is the equivalent of a therapy session or treatment. The patient will undergo as many “therapy” or treatment sessions as necessary until treatment is complete.

### Advantages and Long Felt Need

Using AR/VR, therapists are able to treat a wider variety of patients because they can now form a close relationship with patients outside of their proximity as well as reduce downtime in-between seeing patients. Therapists can also treat patients from the comfort of their own home.

Using AR/VR, the benefits for mental health patients are even greater. Patients are able to receive treatment anywhere, reducing the friction of carving transportation time out of their day to see a therapist (driving, waiting in the lobby, etc.).

Additionally, an AR/VR projection/input of a therapist is able to connect both sides of a therapy session in a more seamless, efficient, and effective way.

Other embodiments are directed to providing tele-health in remote areas for group therapy.

### Figures

In an embodiment of the instant invention, an AR/VR group therapy process method may unfold as follows:

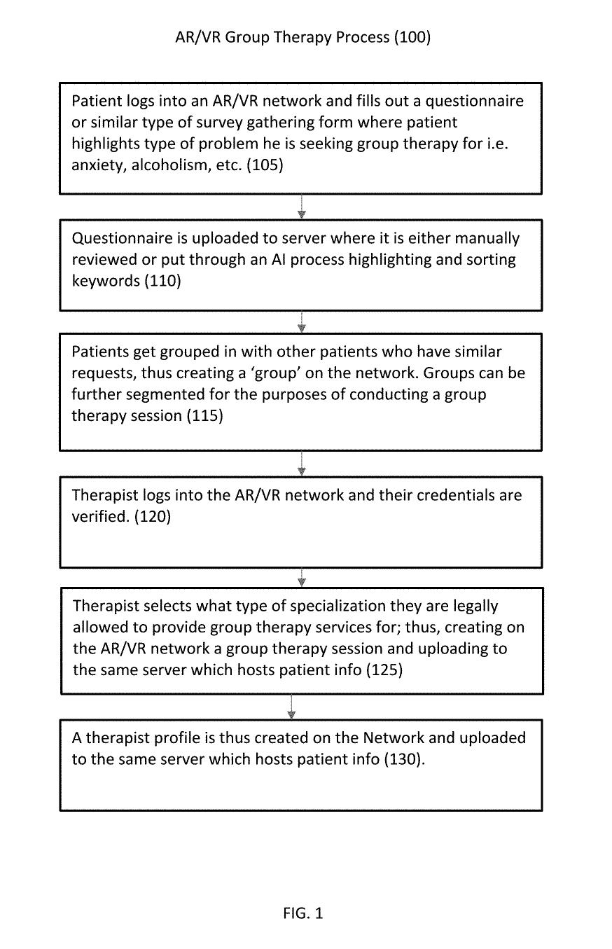

As depicted in FIG. 1, An AR/VR Group Therapy Process (100) may comprise the steps of a patient logging onto an AR/VR network and fills out a questionnaire or similar type of survey gathering form where patient highlights type of problem he is seeking group therapy for i.e. anxiety, alcoholism, etc. (105).

The questionnaire is uploaded to server where it is either manually reviewed or put through an AI process highlighting and sorting keywords (110).

Patients get grouped in with other patients who have similar requests, thus creating a ‘group’ on the network. Groups can be further segmented for the purposes of conducting a group therapy session (115).

A therapist logs into AR/VR network and their credentials are verified (120).

The therapist selects what type of specialization they are legally allowed to provide group therapy services for; thus, creating on the AR/VR network a group therapy session and uploading to the same server which hosts the patient info (125).

A therapist profile is thus created on the AR/VR network and uploaded to the same server which hosts patient info (130)

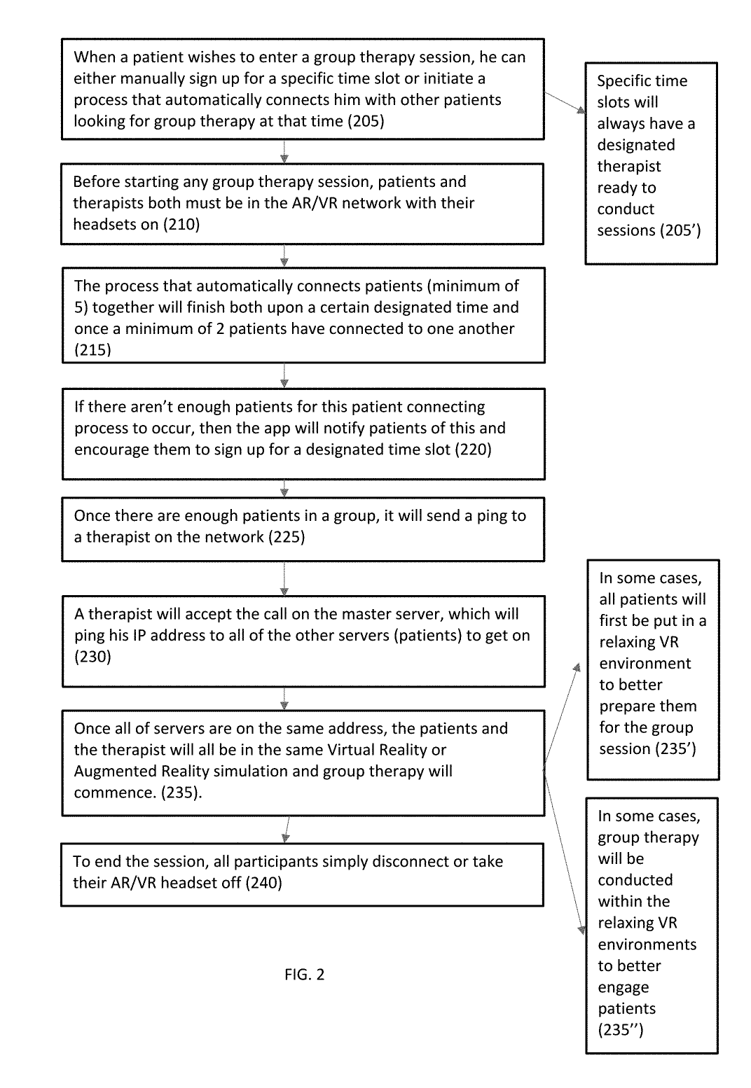

As depicted in FIG. 2, it is contemplated that when a patient wishes to enter a group therapy session, they may either manually sign up for or log into a specific time slot, or initiate a process that automatically connects them with other patients looking for similar group therapy at that time (205).

Specific time slots will always have a designated therapist ready to conduct sessions (205′)

Before starting any group therapy session, patients and therapists both must be in the AR/VR network with their headsets on (210).

The process that automatically connects patients (minimum of 2) together will finish both upon a certain designated time and once a minimum of 2 patients have connected to one another (215).

If there aren't enough patients for this patient connecting process to occur, then the app will notify patients of this and encourage them to sign up for a designated time slot (220).

Once there are enough patients in a group, it will send a ping to a therapist on the network (225).

A therapist will accept the call on the master server, which will ping his IP address to all of the other servers (patients) to get on (230).

Once all of servers are on the same address, the patients and the therapist will all be in the same Virtual Reality or Augmented Reality simulation and group therapy will commence (235).

In some cases, all patients will first be put in a relaxing VR environment to better prepare them for the group session (235′).

In some cases, group therapy will be conducted within the relaxing VR environments to better engage patients.

To end the session, all participants simply disconnect or take their AR/VR headset off (240).

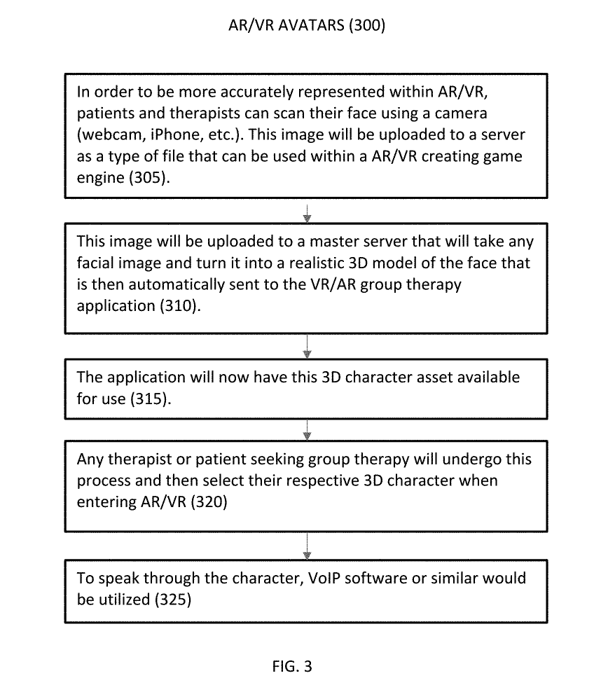

As depicted in FIG. 3, it is contemplated that AR/VR AVATARS may be used to represent a therapist and/or patient within an AR/VR group session environment (300).

In order to be more accurately represented within AR/VR, patients and therapists can scan their face using a camera (webcam, iPhone, etc.) This image will be uploaded to a server as a type of file that can be used within a AR/VR creating game engine (305).

This image will be uploaded to a master server that will take any facial image and turn it into a realistic 3D model of the face that is then automatically sent to the AR/VR group therapy application (310)

The application will now have this 3D character asset available for use (315).

Any therapist or patient seeking group therapy will undergo this process and then select their respective 3D character when entering AR/VR (320)

To speak through the character, VoIP software or similar would be utilized (325).

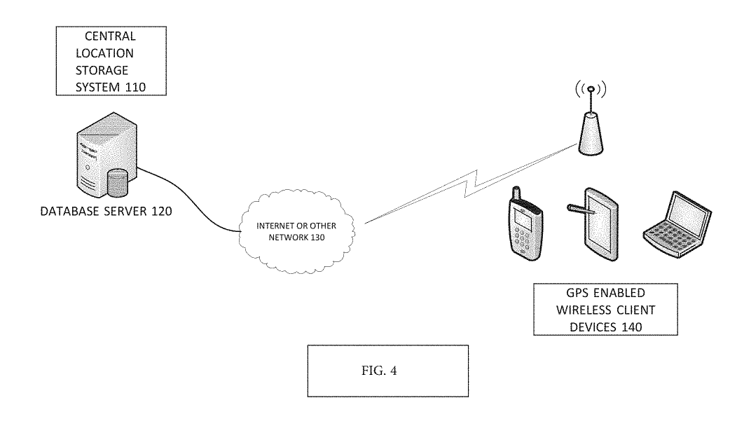

FIG. 4 shows a schematic diagram of one embodiment of a system that can be used to practice aspects of the present invention. The schematic shows a central location storage system 110 and database server 120, which are connected to augmented reality devices 140 via the internet or other network 130. In this manner, augmented reality devices 140 can communicate with the database server 120 via the internet or other network 130. This allows augmented reality devices to be located remote from the server, such that the augmented reality devices can be located at an remote from the server and from a therapist who is using a device that is communication with the augmented reality devices.

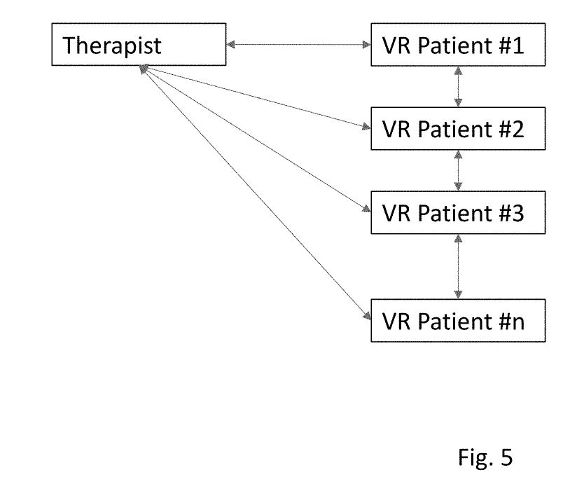

FIG. 5 is a flowchart of a therapist with VR patients such that the therapist and VR patients are in a group therapy environment. In this embodiment, a therapist and at least two patients are present in the group therapy environment, all the way to n patients. The patients are able to see avatars of one another so that they can all interact in a group therapy environment.

In certain embodiments, patients are grouped together, so there are two grouping processes. In one embodiments, patients are grouped with other patients. In other embodiments, patients are grouped with a therapist and a therapist is grouped with patients. In certain embodiments, patients first match with other patients that have similar issues and then those groups are stored and matched with therapists who deal with those issues. Over time, the process will use machine learning to be able to better predict the sorting process, more efficiently matching patients with other patients and therapists with groups.

### Additional Embodiments of the Invention

In certain embodiments, there is a therapist inside an AR/VR environment for group therapy. AR/VR is used to conduct group therapy treatment for mental health, substance abuse, and developmental disorder treatment, among others, because of a variety of reasons:
It gives a single therapist better access to a multitude of patients spread across geographical areas.
It reduces downtime between sessions.
It gives therapists a chance to conduct therapy sessions even when they're on the go/traveling.

AR/VR group therapy also gives patients an easy way to receive treatment from the comfort of their own homes. Patients are still able to receive all the benefits of group therapy: cheaper cost; the sharing of their struggles with similar individuals; ability to identify with others; receiving and giving support to others; getting a variety of viewpoints; and seeing successful models of individuals who have gone through similar experiences—but now they can do so without needing to go into a therapist's office and wait in a long queue.

AR/VR group therapy also takes away some typical disadvantages of group therapy, such as needing to meet at specific times. Group therapy within AR/VR can be conducted anytime as long as a user has a capable headset/device, is able to connect to internet, and can tap into a network of other users and therapists.

Since patients can be plugging into AR/VR group therapy from all over the world, confidentiality is also less of an issue. Typically, group therapy patients come from similar communities, since they all drive to and see the same therapist. Patients can also choose not to be in therapy sessions with those in their proximity.

In certain embodiments, connecting users and therapists together and allowing figures to be represented in VR/AR can be approached from multiple angles:
Option to select a few different avatar characters
Option to select and custom build an avatar character using pre-made selections, which may look similar but not exactly like them.
Option to digitally scan one's face, which gets uploaded to a server that automatically creates 3D models using faces, thus giving a near perfect replica of a person in AR/VR.

The three above are listed in order of increasing difficulty, with number 3 being the most expensive on AR/VR ‘gameplay’ quality.

In terms of data, therapists can better track attention rates via eye movement within AR/VR, such as seeing when a user was more focused or when he/she seemed to zone out.

Patients who have similar issues will be matched up with therapists who are specialists dealing with those issues, thus creating a seamless network of patients and therapists.

In certain embodiments, the process involves the following steps:
Patient signs up for a mental health, substance abuse, developmental disorder issue they'd like to seek help for grouped in with other patients with a similar disorder and an group is created—for instance ‘anxiety group’.
Therapist signs up as someone who specializes in treating patients who have anxiety and the therapist is matched up with anxiety group. Minimum of 2 members needed to conduct a session.
Sessions initiate.
Once signing up for a session, patients need to maintain above a certain percentage of attendance or else they're dropped from the session.
Sessions are ongoing. New members can join existing sessions and older members can try out new sessions as well.

Before patients begin group therapy, they can first be put into a Let's Relax!™ environment created for the purpose of mental wellbeing. This can ease patients and put them in a more conducive mood for therapy.

### Computer Hardware

In certain embodiments of the device, the AR/VR device includes a computer memory, a processor, a headset. In certain embodiments, the AR/VR device is part of a larger system that includes a camera and a mobile device to stream the virtual therapist to the AR/VR device.

Hardware components for augmented and virtual reality are: processor, display, sensors and input devices. Other components include a camera and MEMS sensors such as accelerometer, GPS, and solid state compass.

AR/VR displays can be rendered on devices resembling eyeglasses or lenses, including contact lenses.

Various technologies are used in augmented and virtual reality rendering, including optical projection systems, monitors, handheld devices, and display systems worn on the human body.

### Exemplary Embodiments Described Above

It should be understood that this description is not intended to limit the embodiments. On the contrary, the embodiments are intended to cover alternatives, modifications, and equivalents, which are included in the spirit and scope of the embodiments as defined by the appended claims. Further, in the detailed description of the embodiments, numerous specific details are set forth to provide a comprehensive understanding of the claimed embodiments. However, one skilled in the art would understand that various embodiments can be practiced without such specific details.

Although the features and elements of aspects of the embodiments are described being in particular combinations, each feature or element can be used alone, without the other features and elements of the embodiments, or in various combinations with or without other features and elements disclosed herein.

This written description uses examples of the subject matter disclosed to enable any person skilled in the art to practice the same, including making and using any devices or systems and performing any incorporated methods. The patentable scope of the subject matter is defined by the claims, and can include other examples that occur to those skilled in the art. Such other examples are intended to be within the scope of the claims.

The above-described embodiments are intended to be illustrative in all respects, rather than restrictive, of the embodiments. Thus, the embodiments are capable of many variations in detailed implementation that can be derived from the description contained herein by a person skilled in the art. No element, act, or instruction used in the description of the present application should be construed as critical or essential to the embodiments unless explicitly described as such. Also, as used herein, the article “a” is intended to include one or more items.

All United States patents and applications, foreign patents, and publications discussed above are hereby incorporated herein by reference in their entireties.

What is claimed is:

1. A computer-implemented method for treating patients via group therapy, the method comprising:
providing at least three augmented reality (AR) or virtual reality (VR) devices, the devices each having at least one processor;
creating, via software executing on a processor, an field of view;
overlaying, via software executing on a processor, a therapist within the field of view to create a modified field of view; and
displaying the modified field of view on the at least two devices,
wherein the therapist within the modified field of view provides a therapy to at least two patients suffering from a mental health, behavioral health, or developmental disorder.

2. The computer-implemented method of claim 1, wherein each of the patients in the group of patients wears a wearable device.

3. The computer-implemented method of claim 2, wherein the wearable device is wireless and provides AR/VR content to the patient.

4. The computer-implemented method of claim 1, wherein the group of patients are provided therapy for anxiety, sexual trauma, depression, addiction, and other disorders.

5. The computer-implemented method of claim 1, wherein the method allows users to experience the environment as a group, whereby each patient is in the same environment and receives treatment as a group.

6. The computer-implemented method of claim 1, wherein the patients first experience a relaxing VR environment before starting treatment to better ease them into group therapy.

7. The computer-implemented method of claim 1, wherein the AR/VR environment includes a version of a licensed healthcare professional/therapist in the environment.

8. The computer-implemented method of claim 7, wherein the licensed healthcare professional/therapist is provided live to the group of patients.

9. The computer-implemented method of claim 1, wherein the LHP/therapists and patients include character assets to identical replicas of the people in question.

10. The computer-implemented method of claim 1, wherein all users are able to speak live and be heard in the VR/AR environments.

11. The computer-implemented method of claim 1, wherein there is a network/server by which therapists and patients will be able to connect with each other and enter into VR/AR group therapy sessions.

12. A computer-implemented system for treating patients via group therapy, the system comprising:
a processor; and
software executing on the processor that provides AR/VR content to a group of patients.

13. The system of claim 12, wherein the system includes a wearable wireless device.

14. The system of claim 13, wherein the wireless device includes glasses, traditional head mounted displays, or a wearable medical device such as contact lenses.

15. An augmented reality (AR) or virtual reality (VR) system for treating at least two patient suffering from a mental health, behavioral health, or developmental disorder via group therapy, the system comprising:
at least three augmented reality (AR) or virtual reality (VR) devices, the devices each having at least one processor;
at least one computer-readable storage media storing instructions which, when executed by the at least one processor of each device:
creates a field of view,
overlays a therapist within the field of view to create a modified field of view, and
displays modified field of view on the device,
wherein the therapist within the modified field of view provides a therapy to at least two patients suffering from a mental health, behavioral health, or developmental disorder.
